(19) United States
(12) Patent Application Publication
(10) Pub. No.: US 2018/0228730 A1
(43) Pub. Date: Aug. 16, 2018

(54) USE OF WATER-SOLUBLE POLYMERS BASED ON N-VINYLPYRROLIDONE AND ACRYLIC ACID AS PHARMACEUTICAL ADJUVANTS

(71) Applicant: BASF SE, Ludwigshafen am Rhein (DE)

(72) Inventors: Heiko Alexander Schiffter, Overath (DE); Maximilian Angel, Kasendorf (DE); Karl Kolter, Limburgerhof (DE); Felicitas Guth, Neustadt (DE)

(21) Appl. No.: 15/751,889

(22) PCT Filed: Aug. 17, 2016
(86) PCT No.: PCT/EP2016/069456

§ 371 Date: Feb. 12, 2018

(30) Foreign Application Priority Data
Aug. 21, 2015 (EP) 15181992.7

(51) Int. Cl. A61K 9/16; A61K 9/14; A61K 9/48; A61K 31/4515

(52) U.S. Cl. CPC A61K 9/1635; A61K 31/4515; A61K 9/4866; A61K 9/146

(57) ABSTRACT

Use of a water-soluble polymer having a solubility in water of greater than 5% (m/m) in the pH range of 1 to 13 and which is obtained by free-radically initiated polymerization of a monomer mixture of i) 70 to 90% by weight N-vinylpyrrolidone and ii) 10 to 30% by weight acrylic acid, wherein the sum total of i) and ii) corresponds to 100% by weight, for the formulation of basic active ingredients sparingly soluble in water, wherein the active ingredients in uncharged form or as hydrochloride have a solubility of less than 0.1% (m/m) in water, artificial intestinal juice or gastric juice.

The present invention relates to the use of water-soluble polymers based on N-vinylpyrrolidone and acrylic acid as pharmaceutical auxiliaries, particularly to improve the solubility of basic active ingredients sparingly soluble in water. The invention further relates to solid solutions of basic active ingredients and water-soluble polymers, methods for preparing such solid solutions, polymer salts which can be obtained from such solid solutions and also dosage forms comprising such solid solutions.

Numerous medicaments have a very low solubility in water and can consequently not be absorbed from the gastric and intestinal tract. The result is very low bioavailability.

To improve the bioavailability, different approaches to solve this problem can be pursued.

For medicaments which have a basic group, corresponding salts can be formed by reaction with acids, said salts sometimes having better solubilities. For this purpose, low molecular weight acids or alkalis are generally used. The most common acids are: hydrochloric acid, sulfuric acid, acetic acid, citric acid, tartaric acid, fumaric acid, maleic acid, malonic acid, succinic acid and phosphoric acid. Often, there is barely any difference between the solubility of the medicament acid or base and that of a salt with the specified compounds. The cause of this poor solubility is usually that the salt forms a very stable crystal lattice which is energetically in a favorable state, as a result of which the tendency to dissolve is low. If, additionally, the energy gain as a result of hydration is low, the solubility is further reduced.

A further possibility to overcome the problem of poor solubility is the formation of salts of the sparingly soluble medicaments with polymeric acids or bases. Such salts have hitherto already been produced in principle, although polymers were often used which were not soluble over a large pH range—in particular not in the physiologically relevant range of pH 1-8—or which in solution as acid, base or salt, have a high viscosity. As a result, the release is prevented or at least greatly slowed.

If, however, the polymers have high viscosity in aqueous solution, the active ingredient release from a solid administration form such as, for example, a tablet, is likewise delayed. Upon dissolution of the salt, a gel or a highly viscous solution forms on the surface of the tablet and in the cavities, preventing further penetration of the water into the tablet core and slowing disintegration. This effect and also the reduced diffusion coefficients of the medicament molecules through areas with high viscosity delay the release of the medicament. In this respect, gel-forming polymers are unsuitable for producing rapid-release forms in which a sparingly soluble medicament is to be dissolved quickly and provided to the entire gastric and small intestine surface for absorption.

EP 0211268 describes minoxidil salts with polymeric polyanions which have delayed release and are used for dermal application. Minoxidil is a medicament which comprises 4 groups capable of salt formation and the corresponding polymeric salts were less soluble than the hydrochloride. The numerous groups capable of salt formation greatly reduce the dissociation of the salt and do not improve the solubility compared with the hydrochloride. Oral applications are not described.

U.S. Pat. No. 4,997,643 describes a biocompatible, film-forming delivery system for topical application which comprises a polymeric salt with a carboxyl-group-carrying component. The medicament used is likewise minoxidil, which has the aforementioned special characteristics. Oral applications are not described.

U.S. Pat. No. 4,248,855 claims liquid preparations which comprise salts of basic medicaments and water-insoluble polymers, and which have a slow-release effect. As a result of the use of water-insoluble polymers, the preparations do not exhibit rapid release or high solubility over a large pH range.

It is known from U.S. Pat. No. 5,736,127 that salts may be formed from basic medicaments and polymers obtained by partial hydrolysis of gel-forming polyacrylonitriles. On account of the high molecular weights, the polymers are gel-forming, as a result of which the release of the active ingredients is delayed. Suitability for rapid-release tablets is not stated.

U.S. Pat. No. 4,205,060 describes microcapsules with delayed release which comprise, in the core, a salt of a basic medicament with a carboxyl-group-containing polymer and which is surrounded by a water-insoluble polymer. The carboxyl-group-containing polymer reduces the release of the soluble medicaments used.

Salts of ranitidine with polycarboxylic acids are described in EP 0721785. The polycarboxylic acids bind the ranitidine and are intended to reduce its bitterness. However, low molecular weight salts of ranitidine are readily soluble, meaning that the polycarboxylic acids merely restrict the mobility and diffusion of the ranitidine, meaning that it does not pass so rapidly to the bitter receptors.

To be able to form such polymeric salts, however, the active ingredient must have a certain residual solubility in an aqueous or aqueous-organic system which is often not the case.

U.S. Pat. No. 4,853,439 describes water-soluble complexes of homopolymers or copolymers of N-vinylpyrrolidone with water-insoluble active ingredients. Among the copolymers described are in very general terms those which may comprise acid group-containing monomers such as acrylic acid. Specifically described, however, is only a copolymer with maleic anhydride which, due to physical characteristics such as color, purity and viscosity, is unacceptable for the pharmaceutical user.

H. Uelzmann, Journal of Polymer Science, Vol. XXXIII, pp. 377-379 (1958) describes without details of a possible use relatively higher molecular weight copolymers of acrylic acid and N-vinylpyrrolidone with different molar proportions, wherein such copolymers at molar ratios of 1:1 or 1:2 AA:VP are soluble in water although in concentrated form (50 weight strength). However, when diluted with water to 5 weight strength mixtures for example, the copolymer precipitates out. The copolymers are also insoluble in dilute hydrochloric acid. Such solubility behavior for use for improvement of solubility of pharmaceutical active ingredients is unacceptable.

Another possibility to improve the solubility of sparingly soluble medicaments consists of preparing so-called “solid solutions” of the medicaments in a polymer matrix. In such solid solutions the medicament is distributed in the polymer matrix dispersed at the molecular level. See for example: Janssens S. and Van den Mooter G.: Review: Physical chemistry of solid dispersions, Journal of Pharmacy and Pharmacology 61, 1571-1586 (2009); Vasconcelos et al., Solid dispersions as strategy to improve oral bioavailability of poor water soluble drugs, Drug Discovery Today, Volume 12, Numbers 23/24, 1068-1075, (2006).

Such solid solutions may be obtained from organic solutions of both components by evaporation of the solvent or by melt extrusion. However, some active ingredients are so sparingly soluble that they often cannot be dissolved to a sufficient degree even in those solvents which are acceptable for the technical application. In this case, only melt extrusion remains as alternative mode of preparation in which, however, certain requirements must be imposed on the temperature and shear stability of the components used.

Polymeric active ingredient salts are known from WO 2009/074609 which have a high proportion of acid group-containing monomers. Specifically, polymeric counterions are described comprising at least 80% by weight of carboxyl group-containing monomers such as the acrylic acid. It has been shown, however, that these polymers have disadvantages despite their good solubility in the application. For instance, they have a tendency to decarboxylate under thermal stress. In particular, during the processing in the melt in the extruder, decomposition of the polymers by decarboxylation occurs. They also have disadvantages with respect to storage stability. Due to the high carboxyl group content, there is a greater risk of interaction of the excess acid groups and thus damage to the active ingredients at sensitive binding sites.

Another problem is that for water-sensitive active ingredients, a conversion into hydrochlorides is not recommended.

Lack of stability of the active ingredient, or of the formulation auxiliaries or active ingredient formulation with respect to degradation or discoloration of the formulations, is not acceptable from the perspective of a pharmaceutical company, no more than in the food supplement or animal feed sector or in cosmetics.

It was an object of the present invention to find active ingredient formulations which help to avoid the disadvantages of the prior art and, with good stability, to permit a more rapid release of the active ingredient compared to the corresponding hydrochloride salt. Furthermore, the formulations sought should also be soluble irrespective of the pH. In addition, the formulations should have no concentration-dependent miscibility gaps in dilute solutions.

Accordingly, the use of a water-soluble polymer having a solubility in water of greater than 5% (m/m) in the pH range of 1 to 13 and which is obtained by free-radically initiated polymerization of a monomer mixture of i) 70 to 90% by weight N-vinylpyrrolidone and ii) 10 to 30% by weight acrylic acid, wherein the sum total of i) and ii) corresponds to 100% by weight, has been found for the formulation of basic active ingredients sparingly soluble in water, wherein the active ingredients in uncharged form or as hydrochloride have a solubility of less than 0.1% (m/m) in water, artificial intestinal juice or gastric juice.

According to a preferred embodiment, the invention relates to the use of a water-soluble polymer having a solubility in water of greater than 5% (m/m) in the pH range of 1 to 13 and which is obtained by free-radically initiated polymerization of a monomer mixture of i) 70 to 90% by weight N-vinylpyrrolidone and ii) 10 to 30% by weight acrylic acid, wherein the sum total of i) and ii) corresponds to 100% by weight, for the formulation of basic active ingredients sparingly soluble in water, which in uncharged form or as hydrochloride have a solubility of less than 0.1% (m/m) in water, artificial intestinal juice or gastric juice, for the purposes of solubilizing the basic active ingredients in an aqueous medium.

Solubilization according to the invention signifies that the water solubility of the active ingredients in the formulation is greater than the solubility of the corresponding hydrochloride salts.

According to a further embodiment, the water-soluble polymers according to the invention are used as binders in the preparation of solid dosage forms of basic active ingredients sparingly soluble in water, which in uncharged form or as hydrochloride have a solubility of less than 0.1% (m/m) in water, artificial intestinal juice or gastric juice.

According to a particularly preferred embodiment, the invention relates to solid solutions of basic active ingredients sparingly soluble in water, which in uncharged form or as hydrochloride have a solubility of less than 0.1% (m/m) in water, artificial intestinal juice or gastric juice, and a water-soluble polymer having a solubility in water of greater than 10% (m/m) in the pH range of 1 to 13 and which is obtained by free-radically initiated polymerization of a monomer mixture of i) 70 to 90% by weight N-vinylpyrrolidone and ii) 10 to 30% by weight acrylic acid, wherein the sum total of i) and ii) corresponds to 100% by weight.

The formulations according to the invention are water-soluble and can dissolve completely in water under standard conditions to form optically clear solutions. The solid solutions have a solubility in water under standard conditions which is greater than that of the corresponding hydrochlorides. Standard conditions mean 20+/−5° C. and standard atmospheric pressure of 1013.25 hPa.

The formulations according to the invention lead not only to an increased solubility but also to a more rapid release of active ingredient in customary release media such as water, artificial gastric or intestinal juice. In this manner, a higher bioavailability is generally also achieved.

Furthermore, methods for preparing the solid solutions and also use thereof in pharmaceutical dosage forms have been found.

“Formulation” signifies a physical mixture, which enables the administration as medicament and which improves bioavailability. Essentially, there are no strong intermolecular interactions between the water-soluble polymers and the basic active ingredients. “No strong intermolecular interactions” signifies that only weak interactions occur as interactions between the molecules such as van der Waals interactions, hydrogen bonding or complex bonding.

Suitable active ingredients in the context of the invention are basic active ingredients sparingly soluble in water.

“Basic” signifies that the active ingredient is capable of forming cations and “sparingly soluble in water” signifies that the active ingredients in the context of the invention are those which in uncharged form or as hydrochloride have solubilities of less than 0.1% (m/m) in water, artificial intestinal juice or gastric juice (at 20+/−5° C. and standard atmospheric pressure of 1013.25 hPa).

In principle, all pharmaceutical active ingredients, nutraceuticals, food or feed additives having a sufficient basicity are suitable.

Preferred basic active ingredients have at least one and at most two groups capable of salt formation.

In particular, the basic active ingredients are pharmaceutical active ingredients.

According to the invention, it is also possible to dissolve medicaments for which neither the neutral form nor the corresponding low molecular weight salts are soluble in water. For these medicaments, the dissolution in the gastric and intestinal tract is very slow and thus limiting for the absorption, as a result of which low bioavailability often results (according to the Biopharmaceutical Classification System: Class II active ingredients (Amidon et al., Pharm. Res. 12, 413-420)).

The pharmaceutical active ingredients here may be from any indication field.

Examples which may be mentioned here include antihypertensives, vitamins, cytostatics, especially taxol, anesthetics, neuroleptics, antidepressants, antibiotics, antimycotics, fungicides, chemotherapeutics, urologics, platelet aggregation inhibitors, sulfonamides, spasmolytics, hormones, immunoglobulins, sera, thyroid therapeutics, psychopharmaceuticals, Parkinson's drugs and other antihyperkinetics, ophthalmics, neuropathy preparations, calcium metabolism regulators, muscle relaxants, narcotics, antilipemics, liver therapeutics, coronary drugs, cardiac drugs, immunotherapeutics, regulatory peptides and their inhibitors, hypnotics, sedatives, gynecological drugs, gout remedies, fibrinolytics, enzyme preparations and transport proteins, enzyme inhibitors, emetics, weight-loss drugs, perfusion promoters, diuretics, diagnostics, corticoids, cholinergics, biliary therapeutics, antiasthmatics, broncholytics, beta-receptor blockers, calcium antagonists, ACE inhibitors, arteriosclerosis remedies, antiphlogistics, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists or antiviral active ingredients or active ingredients for the treatment of HIV infections and AID syndrome.

The water-soluble polymers used according to the invention are all water-soluble independent of pH in the entire pH range of pH 1 to pH 13. Water-soluble signifies that at least 5% (m/m) polymer is clearly soluble in water. No turbidity should be apparent optically in the clear solutions.

In particular, the water-soluble polymers have a solubility in water of greater than 10% (m/m) in the pH range of 3 to 11.

All data on the water solubility refer, in the case of all water-soluble polymers used in accordance with the invention to standard conditions of 20+/−5° C. and standard atmospheric pressure of 1013.25 hPa. The datum (m/m) denotes mass fractions.

The water-soluble polymers according to the invention have no miscibility gaps in the concentration range of 5% by weight to 50% by weight, based on polymer in solvent, in water and in 0.08 M hydrochloric acid as solvents. “No miscibility gaps” signifies that the polymers are clearly soluble in the solvent and that no turbidity should thus be apparent optically.

The water-soluble polymers used in accordance with the invention form optically clear solutions in water which are also storage-stable. They have no sediment even after storage for six months in the concentration range mentioned above at 40° C. and standard pressure. “No sediment” signifies that less than 1% by weight of the polymer used in the preparation of the solution precipitates out of the solution. Preference is given to water-soluble polymers which, on storage under the conditions stated in an aqueous dissolution medium, precipitate less than 0.1% by weight, based on the amount of water-soluble polymer used.

All water-soluble polymers mentioned are non-gel-forming in solvents, particularly in water. Non-gel-forming signifies that they do not form three-dimensional networks and therefore comprise no pores into which solvent molecules could penetrate.

The water-soluble polymers have Fikentscher K-values, measured in a 5% by weight aqueous solution, of less than 30, particularly preferably less than 20. This K-value is a characteristic of the viscosity of the solution, which in turn represents a measure of the molecular weight of these polymers.

The glass transition temperatures calculated according to the Fox equation are in the range of 140 to 160° C.:

$$\frac{1}{{T}_{G}} = {\sum }_{i}^{n} {x}_{i} \frac{1}{{T}_{G , i}}$$

xᵢ=mass fraction of the comonomer in the polymer

T_{G,i}=glass transition temperature of the homopolymer of the corresponding comonomer

T_{G}=glass transition temperature of the copolymer

The glass transition temperatures may also be measured by differential scanning calorimetry at a heating rate of 20 K/min and are in the range of 130 to 170° C.

As already mentioned, suitable water-soluble polymers for water-soluble formulations of active ingredients sparingly soluble in water consisting of a basic active ingredient, which in uncharged form or as hydrochloride has a solubility of less than 0.1% (m/m) in water, artificial intestinal juice or gastric juice, are those polymers having a solubility in water of greater than 10% (m/m) over the entire pH range of 1 to 13 and which are obtained by free-radically initiated polymerization of a monomer mixture of i) 70 to 90% by weight N-vinylpyrrolidone and ii) 10 to 30% by weight acrylic acid, wherein the sum total of i) and ii) corresponds to 100% by weight.

Preference is given to water-soluble polymers having a solubility in water of greater than 10% (m/m) over the entire pH range of 1 to 13 and which are obtained by free-radically initiated polymerization of a monomer mixture of i) 75 to 85% by weight N-vinylpyrrolidone and ii) 15 to 25% by weight acrylic acid, wherein the sum total of i) and ii) corresponds to 100% by weight.

Particular preference is given to using a water-soluble polymer having a solubility in water of greater than 10% (m/m) over the entire pH range of 1 to 13 and which is obtained by free-radically initiated polymerization of a monomer mixture of i) 80% by weight N-vinylpyrrolidone and ii) 20% by weight acrylic acid, wherein the sum total of i) and ii) corresponds to 100% by weight.

The polymers can be prepared in a conventional manner per se by free-radical polymerization. The polymerization is preferably carried out as a solution polymerization in organic solvents, preferably in alcohols, particularly in isopropanol. Such methods are known per se to those skilled in the art. Suitable initiators are, for example, organic peroxides such as tertiary-butyl perpivalate or alcohol-soluble azo starters.

The polymerization may be conducted at temperatures of 50 to 130° C. and pressures of 0.1 to 1.5 MPa.

It may also be recommended to carry out the polymerization in the presence of chain transfer agents, for example sodium hypophosphite.

The polymerization can be carried out continuously or as a batch process, the polymers preferably being obtained via a feed process.

According to one embodiment of the invention, the sodium salt of the copolymer is firstly prepared by free-radical copolymerization of N-vinylpyrrolidone and sodium acrylate, which can then be converted into the free acid copolymer by ion exchange.

The conversion of the polymer solutions into the solid form may also be carried out by conventional drying processes such as spray-drying, freeze-drying or roller drying.

According to a particularly preferred embodiment, the polymers have Fikentscher K-values, measured in a 5% strength by weight aqueous solution, in the range from 10 to less than 20.

During the preparation of the polymers according to the invention, it is preferably to be ensured that these have no low molecular weight anions such as, for example, chloride, sulfate, etc. which can lead to sparingly soluble salts with active ingredients.

Preference is given to using the water-soluble polymers thus obtained for preparing solid solutions with basic active ingredients sparingly soluble in water.

According to one embodiment of the invention, the solid solutions according to the invention can be prepared by means of the solvent method.

The active ingredient and the polymer are dissolved in organic solvents or solvent mixtures and the solution is then dried. The dissolution can also take place at elevated temperatures (30-200° C.) and under pressure.

Suitable organic solvents are dimethylformamide, methanol or mixtures thereof and mixtures with isopropanol, tetrahydrofuran, acetone or ethyl acetate.

In principle, all types of drying are possible, such as, for example, spray-drying, fluidized-bed drying, drum drying, freeze-drying, vacuum drying, belt drying, roller drying, carrier-gas drying, evaporation etc.

According to a preferred embodiment of the invention, the solid solutions are prepared by melt processes. The active ingredient is mixed with the polymer. By heating to temperatures of 50-250° C., the production of the solid solution takes place. Here, temperatures above the glass transition temperature of the polymer or the melting point of the active ingredient are advantageous. By adding a softening auxiliary, such as, for example, water, organic solvent, customary organic softeners, it is possible to correspondingly reduce the processing temperature. Of particular advantage here are auxiliaries which can afterwards be very easily evaporated off again, i.e. having a boiling point below 180° C., preferably below 150° C.

According to a preferred embodiment, this type of preparation is carried out in a screw extruder. Which process parameters must be individually adjusted here can be determined by those skilled in the art by simple experiments in the scope of his or her conventional specialist knowledge.

According to a preferred embodiment, softeners are added during the melting. Preferred softeners are citric esters such as triethyl citrate or acetyl tributyl citrate, glycol derivatives such as polyethylene glycol, propylene glycol or poloxamers; castor oil and mineral oil derivatives; sebacate esters such as dibutyl sebacate), triacetin, fatty esters such as glycerol monostearate or stearyl alcohol or vitamin E TPGS (tocopherol glyceryl succinate), particularly tocopherol glyceryl succinate or polyethylene glycol 1500. The softeners may be used in amounts of 0.1 to 40% by weight, preferably 1 to 20% by weight, based on the polymer.

The solid solutions generated are X-ray amorphous. The amorphous state can be established by X-ray diffraction. The so-called “X-ray amorphous” state of the solid solutions signifies that the crystalline proportion is less than 5% by weight.

The amorphous state can also by investigated with the aid of a DSC thermogram (Differential Scanning Calorimetry). The solid solutions according to the invention have no melting peaks but only a glass transition temperature, which depends also on the type of active ingredient used in the solid solutions according to the invention. The glass transition temperatures, measured at a heating rate of 20K/min, are predominantly in the range of 50 to 170° C.

In the course of preparation of the solid dosage forms according to the invention, customary pharmaceutical auxiliaries may optionally be processed at the same time. These take the form of substances of the class of fillers, softeners, solubilizers, binders, silicates and also disintegrants and adsorbents, lubricants, flow agents, dyes, stabilizers such as antioxidants, wetting agents, preservatives, release agents, flavorings or sweeteners, preferably fillers, softeners and solubilizers.

The fillers added can be e.g. inorganic fillers such as oxides of magnesium, aluminum or silicon, titanium carbonate or calcium carbonate, calcium phosphates or magnesium phosphates or organic fillers such as lactose, sucrose, sorbitol or mannitol.

Suitable softeners are, for example, triacetin, triethyl citrate, glycerol monostearate, low molecular weight polyethylene glycols or poloxamers.

Suitable additional solubilizers are surface-active substances having an HLB value (Hydrophilic Lipophilic Balance) greater than 11, for example hydrogenated castor oil ethoxylated with 40 ethylene oxide units (Cremophor® RH 40), castor oil ethoxylated with 35 ethylene oxide units (Cremophor eL), polysorbate 80, poloxamers or sodium lauryl sulfate.

The lubricants used may be stearates of aluminum, calcium, magnesium and tin and also magnesium silicate, silicones and the like.

The flow agents used may be, for example, talc or colloidal silicon dioxide.

Suitable binders are, for example, microcrystalline cellulose.

The disintegrants used may be crosslinked polyvinylpyrrolidone, crosslinked sodium carboxymethylcellulose or crosslinked sodium carboxymethyl starch. All substances having antioxidant properties can function as stabilizers, such as ascorbic acid, butylhydroxytoluene or tocopherol.

Dyes are, e.g. iron oxides, titanium dioxide, triphenylmethane dyes, azo dyes, quinoline dyes, indigotine dyes, carotenoids, in order to color the dosage forms, and opacifiers such as titanium dioxide or talc in order to increase the transparency and to save on dyes.

The formulations according to the invention can be formulated to give many different administration forms such as, for example, tablets, capsules, granules, powders, drug delivery systems, solutions, suppositories, transdermal systems, creams, gels, lotions, injection solutions, drops, juices, syrups.

The release can be slowed by the addition of thermoplastic release-slowing agents such as polyvinyl acetate homopolymers or formulations of such polyvinyl acetate homopolymers, also by the known delayed release polymers Eudragit® of RS, RL or NE or NM type, based on acrylate.

In this manner, reliable slow-release forms of poorly soluble medicaments can be produced.

The formulations according to the invention have an excellent processability to dosage forms, particularly with regard to the tabletability. Consequently, it is possible to produce tablets with a diameter of 10 mm and 300 mg in weight which have a fracture resistance of more than 200 N. The water-soluble polymer acts simultaneously as binder and imparts enormous plasticity to the tablet formulation.

Due to the higher solubility, the bioavailability is likewise higher and also much more reproducible, i.e. there is less interindividual variability.

The solid solutions may also be processed at higher temperatures by melt extrusion without leading to changes in the acid number.

### EXAMPLES

### Abbreviations/Methods

X-Ray Diffractometry

Measuring instrument: D8 Advance diffractometer equipped with 9-tube sample changer (Bruker/AXS).

Measurement type: θ-θ geometry in reflection

2 theta angle range: 2-80°

Step width: 0.02°

Measurement time per angular step: 4.8 s

Divergence slit: Göbel mirror with 0.4 mm inserted aperture

Antiscattering Slit: Soller slit

Detector: Sol-X detector

Temperature: room temperature

Generator setting: 40 kV/50 mA

The turbidity was measured according to ISO 7027 by measuring the ratio of scattered light and transmission.

The glass transition temperatures were determined at a heating rate of 20K/min, weight: 8.5 mg. Percent data relate to the percent by weight unless otherwise specified.

### Example 1

Copolymer of 80% by weight N-vinylpyrrolidone (VP) and 20% by weight acrylic acid (AA)

### Apparatus:

2 l HWS pot equipped with anchor stirrer, reflux condenser, nitrogen inlet (via liquid level) and temperature-controlled oil bath. 2 feed vessels each 1000 ml. Temperature measurement in the polymerization vessel and in the oil bath via Pt100 sensor in each case.

| Feeds | Amount | Starting material |
|---|---|---|
| Initial charge | 210.0 g | isopropanol |
| | 30.0 g | N-vinylpyrrolidone |
| | 10.6 g | Part of feed 2 |
| Feed 1 | 399.0 g | isopropanol |
| | 210.0 g | N-vinylpyrrolidone |
| | 60.0 g | Acrylic acid |
| Feed 2 | 100.0 g | isopropanol |
| | 6.0 g | tert-butyl perpivalate, 75% strength |

The initial charge was heated to 75° C. internal temperature under a gentle nitrogen stream. On reaching 75° C. internal temperature, the part amount of feed 2 was added. Feed 1 and the residual amount of feed 2 were then started. Feed 1 was metered in over 6 hours and the residual amount of feed 2 was metered in over 9 hours.

The reaction mixture was then subjected to another steam distillation in order to displace the isopropanol solvent.

| | |
|---|---|
| Solids content SC [% by wt.] | 30.5 |
| K-value (5% strength in water) | 16.5 |
| FNU value (5% strength in water) | 0.7 |
| Tg (° C.) | 165 (measured by differential |
| | scanning calorimetry DSC; calculated |
| | 150° C., see above) |
| Isopropanol (ppm) | 2800 |

Appearance: water-clear low viscosity aqueous solution.

### Example 2

This was carried out as in Example 1, except feed 1 was metered in over 4 hours and the residual amount of feed 2 over 6 hours.

After feed 2 was complete, the polymerization was continued for 1 hour at an internal temperature of 75° C.

| | |
|---|---|
| SC (% by weight) | 30.8 |
| K-value (5% strength in water) | 17.8 |
| FNU value (5% strength in water) | 0.6 |
| Tg (° C.) | 164 (DSC) |
| Isopropanol (ppm) | 2600 |

Appearance: water-clear low viscosity aqueous solution.

### Example 3

Copolymer of 70% by Weight VP and 30% by Weight AA

The preparation was carried out analogously to Example 1, wherein feed 1 comprised 180 g of N-vinyl pyrrolidone and 90 g of acrylic acid.

| | |
|---|---|
| SC (% by weight) | 30.4 |
| K-value (5% strength in water) | 15.8 |
| FNU value (5% strength in water) | 1.5 |
| Tg (° C.) | 144 (calculated) |
| Isopropanol (ppm) | 2200 |

Appearance: water-clear low viscosity aqueous solution.

### Example 4

Copolymer of 90% by Weight VP and 10% by Weight AA

The preparation was carried out analogously to Example 1, wherein feed 1 comprised 240 g of N-vinyl pyrrolidone and 30 g of acrylic acid.

| | |
|---|---|
| SC (% by weight) | 30.3 |
| K-value (5% strength in water) | 16.7 |
| FNU value (5% strength in water) | 0.6 |
| Tg (° C.) | 156 (calculated) |
| Isopropanol (ppm) | 2500 |

Appearance: water-clear low viscosity aqueous solution.

### Example 5

Copolymer of 85% by Weight VP and 15% by Weight AA

The preparation was carried out analogously to Example 1, wherein feed 1 comprised 225 g of N-vinyl pyrrolidone and 45 g of acrylic acid.

| | |
|---|---|
| SC (% by weight) | 30.5 |
| K-value (5% strength in water) | 16.4 |
| FNU value (5% strength in water) | 0.7 |
| Tg (° C.) | 153 (calculated) |
| Isopropanol (ppm) | 2100 |

Appearance: water-clear low viscosity aqueous solution.

### Comparative Example

Copolymer of 50% by Weight VP and 50% by Weight AA

The preparation was carried out analogously to Example 1, wherein feed 1 comprised 120 g of vinylpyrrolidone and 150 g of acrylic acid.

| | |
|---|---|
| SC (% by weight) | 31.5 |
| K-value (5% strength in water) | 12.8 |
| FNU value (5% strength in water) | 3.1 |
| Tg (° C.) | 132 (calculated) |
| Isopropanol (ppm) | 3000 |

The polymer thus obtained having an FNU value of 3.1 was no longer “water-clear” on visual inspection but slightly cloudy.

### Example 6

Preparation of a Solid Solution by Solution Process

1250 g of copolymer VP/AA (80/20) were dissolved together with 416.7 g of haloperidol (base) in 5999.3 g of a solvent mixture consisting of 50% (2999.7 g) ethanol and 50% (2999.7 g) isopropanol at room temperature with stirring. The organic solution had a total solids content of 21.7%. The solution was then spray-dried in a laboratory spray-drier under the following conditions:

Drying gas: Nitrogen; 30 Nm3/h

Inlet temperature: 155° C.

Outlet temperature: 75° C.

Atomization nozzle: 1.4 mm dual component nozzle

Atomizing gas/atomizing pressure: Nitrogen/2 bar abs.

Liquid flow rate: 977.9 g/h

Product separator: cyclone

The table below gives an overview of the properties of the spray-dried polymeric haloperidol-VP/AA salt after spray-drying of the organic solution (ethanol:isopropanol 1:1).

| | |
|---|---|
| Residual moisture content (measured at | 1.75% |
| 105° C.) | |
| Medicament content (measured, | 24.5% |
| UV/VIS at 248 nm) | |
| Medicament state (XRD) | X-ray amorphous |
| Glass transition temperature | 129° C. (no melting peak) |

Neither in the DSC thermogram nor in the X-ray diffractometry were crystalline active ingredient fractions seen. It can therefore be deduced from this that it is in the form of an amorphous powder. The release of the active ingredient from the spray-dried polymeric salt was carried out in demineralized water. The initial weight was calculated on 100 mg of haloperidol per 250 ml of release medium. The polymeric salt or the crystalline substance correspondingly weighed out were filled into hard gelatine capsules. The following table shows the results of the release compared to the crystalline medicament.

| | | |
|---|---|---|
| Release of polymeric haloperidol-VP/AA salt | 0 min | 0% |
| (prepared from organic solution) in demineralized | 2 min | 1.4% |
| water. | 4 min | 62.5% |
| | 6 min | 84.8% |
| | 8 min | 89.0% |
| | 10 min | 95.4% |
| | 30 min | 98.3% |
| | 60 min | 98.6% |
| | 120 min | 98.6% |
| Crystalline haloperidol in demineralized water | 0 min | 0.0% |
| | 2 min | 0.1% |
| | 4 min | 0.2% |
| | 6 min | 0.3% |
| | 8 min | 0.4% |
| | 10 min | 0.6% |
| | 30 min | 1.5% |
| | 60 min | 1.8% |
| | 120 min | 2.3% |

Preparation of the solid solution by the melt process: A polymer according to Example 1 with various active ingredients was processed using a twin screw extruder: Pharma Extruder, Haake PolyLab OS, RheoDrive 7 (measuring system); Haake Rheomex OS, PTW 16 mm (measuring attachment); gravimetric dosage unit Brabender twin-screw feeder type DDSR 20, Software AIO-Control

Screw type L/D 40

Nozzle type: 3 mm, nozzle hole

Speed of rotation: 200/min

Torque: 50 Nm

Metered addition: 1 kg/h

Cooling belt for Extruder type 557-2680 (Thermo Fischer)

Granulator Thermo Electron type 554-1345

Tube Mill (Model Tube Mill control, IKA); grinding of the extrudate fragments at 25 000 rpm

The respective extrusion temperature is listed in the following table.

| Sample | | | Extrusion temperature | | | | |
|---|---|---|---|---|---|---|---|
| identifier | Formulation | | Zone 1 | Zone 2 | Zone 3 | Zone 4-9 | Zone 10 |
| Sample 1 | VP/AA (80/20) | 100% | 50° C. | 80° C. | 195° C. | 270° C. | 250° C. |
| Sample 2 | VP/AA (80/20) | 100% | 50° C. | 80° C. | 170° C. | 300° C. | 230° C. |
| Sample 12 | VP/AA (80/20) | 90% | 50° C. | 80° C. | 170° C. | 225° C. | 190° C. |
| | Kolliphor TPGS | 10% | | | | | |
| Sample 13 | VP/AA (80/20) | 90% | 50° C. | 80° C. | 170° C. | 225° C. | 190° C. |
| | PEG 1500 | 10% | | | | | |
| Sample 14 | VP/AA (80/20) | 85% | 50° C. | 80° C. | 170° C. | 225° C. | 190° C. |
| | Kolliphor TPGS | 10% | | | | | |
| | Loperamide (base) | 5% | | | | | |
| Sample 15 | VP/AA (80/20) | 80% | 50° C. | 80° C. | 170° C. | 225° C. | 190° C. |
| | Kolliphor TPGS | 10% | | | | | |
| | Loperamide (base) | 10% | | | | | |
| Sample 16 | VP/AA (80/20) | 85% | 50° C. | 80° C. | 170° C. | 225° C. | 190° C. |
| | PEG 1500 | 10% | | | | | |
| | Loperamide (base) | 5% | | | | | |
| Sample 17 | VP/AA (80/20) | 80% | 50° C. | 80° C. | 170° C. | 225° C. | 190° C. |
| | PEG 1500 | 10% | | | | | |
| | Loperamide (base) | 10% | | | | | |

Solubilization

The active ingredient release from samples 14, 15, 16 and 17 were tested in a release device with automatic spectroscopic measurement (00501FEXXX-D). The extrudates having 5% content of active ingredient were added directly to the release medium at the beginning of the experiment. The extrudates having 10% content of active ingredient were mixed with 15% (m/m) Avicel PH 101 (microcrystalline cellulose) and 15% (m/m) Kollidon CL (disintegrant). The mixtures were filled into hard gelatine capsules and released therefrom.

Procedure for release (2 hours):

Conditions 0-2 hours: Medium:
250 ml of demineralized water;
paddle stirrer 50 rpm;
temperature 37° C.
content determination UV/VIS with 1 cm path length cuvette
active ingredient amount 250 mg

The results of the releases are compiled in the following tables and diagrams. The release is complete in all cases. The crystalline loperamide (base) showed no dissolution at all in the release medium or no amount of dissolved loperamide could be detected with the spectroscopic method used.

| | Released loperamide [%] | | | |
|---|---|---|---|---|
| | | Sample 15 | | Sample 16 |
| | Sample 14 | 10% | Sample 16 | 10% |
| Time | 5% Loperamide | Loperamide | 5% Loperamide | Loperamide |
| [min] | TPGS | TPGS | PEG 1500 | PEG 1500 |
| 0 | 0.0% | 0.0% | 0.0% | 0.0% |
| 2 | 57.2% | 5.7% | 58.2% | 0.3% |
| 4 | 86.4% | 20.0% | 75.7% | 27.6% |
| 6 | 92.8% | 48.7% | 81.5% | 33.1% |
| 8 | 95.4% | 69.6% | 84.5% | 47.2% |
| 10 | 97.0% | 76.6% | 87.0% | 58.5% |
| 30 | 101.6% | 90.8% | 96.7% | 89.0% |
| 60 | 100.3% | 95.0% | 99.3% | 93.4% |
| 120 | 97.6% | 98.0% | 101.9% | 96.1% |

Tablet Preparation

The polymeric salt having a haloperidol content of 24.5% (m/m), prepared by spray-drying in Example 6, was used for preparing tablets.

Formulation:

| | | |
|---|---|---|
| Ludipress | 72.1% | 360.5 mg |
| Polymeric salt (according to example | 20.4% | 102.0 mg |
| 6, haloperidol content 24.5%) | | |
| Aerosil 200 | 2.0% | 10.0 mg |
| Magnesium stearate | 0.5% | 2.5 mg |
| Kollidon CL | 5.0% | 25.0 mg |
| | 100.0% | 500.0 mg |

The tabletting was carried out using a Korsch XP1 Exzenter tablet press, curved 11 mm punch, at a compression force of: 10 kN. The release of the active ingredient from the tablets prepared was carried out in demineralized water. The initial weight was calculated based on 100 mg of haloperidol per 250 ml of release medium, corresponding to 4 tablets per 250 ml of release medium. The following table shows the results of the release compared to the crystalline medicament.

| | | |
|---|---|---|
| Release of polymeric haloperidol-VP/AA | 0 min | 0% |
| salt (spray-dried from organic solution, | 2 min | 5.4% |
| formulated and compressed into tablets) | 4 min | 12.9% |
| in demineralized water | 6 min | 20.7% |
| | 8 min | 27.8% |
| | 10 min | 34.5% |
| | 30 min | 82.2% |
| | 60 min | 97.4% |
| | 120 min | 97.9% |

Determination of the Binding Type in a Solid Solution

Here, an inventive solid solution of loperamide and the copolymer according to example 1, prepared as described in example 6, was compared with a solid salt of loperamide and the identical copolymer and also with a solid solution of loperamide and PVP K17. The solid solution of loperamide and copolymer was obtained by evaporation of a solution of the substances in THF/methanol. The solid solution of loperamide and PVP was obtained as follows: 1 part loperamide (base) and 9 parts PVP K17 were dissolved in DMF. The solids concentration was 25% (m/m). The solution was poured onto a rubber sheet and dried for 48 hours in a vacuum drying cabinet at 50° C. and 10 mbar. The solid solution obtained was then removed from the rubber sheet and ground to a powder in a tube mill (Model Tube Mill control, IKA) at 25 000 rpm.

Reaction Calorimetry

A Nano ITC (Isothermal Titration Calorimeter) from TA Instruments was used for the investigations. The respective polymer was initially charged in the measuring cell. Loperamide was successively added thereto. Once a reaction takes place, the temperature of the sample changes. This temperature difference was registered and compensated via a Peltier element. The electrical energy required for the compensation was recorded. This amount of energy is identical to the amount of heat of reaction produced or consumed.

The two cells, measuring cell and reference cell, are embedded in a highly stable temperature-controlled bath (+−0.0002 K at 25° C.). Stirring was provided during the experiment.

Due to the low amounts of heat which are measured here, it is necessary to carry out reference measurements. In these cases, the dilution reaction of the polymers. The amount of heat measured during the dilution reaction was subtracted from the original measurement. The value obtained is the enthalpy of reaction of the active ingredient-polymer reaction.

To determine the enthalpy of reaction, the polymers were each prepared at a concentration of 3 g/L and 1 ml initially charged in each case. The loperamide base was prepared at a concentration of 25 mg/l and added in 5 μl steps. After each addition, there was a pause of 600 s until the reaction had ended. Measurement was at 25° C. A water/ethanol mixture (9:1) served as solvent.

The polymers were initially charged in large excess so that it was ensured that each molecule added could find a corresponding reaction partner.

Solution Calorimetry

This investigation was carried out using a TAM III with SolCal insert from TA Instruments. The solvent was initially charged therein. The sample to be dissolved was hermetically sealed in a glass ampoule. The ampoule was introduced into the solvent and the temperature equilibrated. The solution calorimetry was carried out at 25° C. (nominal temperature).

In a next step, the operating temperature was brought to a temperature in the range of 0.3K below the nominal temperature. Then, the equalization of the operating temperature to the nominal temperature was measured, the curve shape was determined from, and was calibrated by specific energy input (electrical energy=thermal energy). The formulations were weighed (20-140 mg) into a crushing ampoule and introduced into 100 mL of a mixture of water/ethanol in a ratio by volume of 9:1.

The ampoule was then broken on a spike in the measuring cell. The substance to be measured was thereby released in the solvent. After subtraction of the baseline of the temperature profile and the conversion of the temperature to the corresponding amount of heat, a heat flow curve of amount of heat vs. time was obtained. After integration of the curve and reference to the molecular weights, the enthalpy of solution of the substance to be investigated was obtained.

The heat of dissolution of all samples was determined in duplicate. The enthalpy of the polymer, which was determined in a separate measurement, was subtracted proportionally from the heat of solution of the formulation.

The table below shows the results of the measurements of the enthalpy of solution of the individual loperamide-polymer formulations. Column A shows is the total amount of heat Q_{Tot} for the respective formulation. Column B shows the proportional amount of heat of the polymer Q_{poly}.

The difference gives the amount of heat Q_{API} of the active ingredient based on the total initial weight (column C) or based on the proportion of the active ingredient (column D). The value in column E estimates the enthalpy of solution or enthalpy of binding (delta H) of the loperamide.

| | A | B | C | D | E |
|---|---|---|---|---|---|
| | Qₜₒₜ | Q_{Poly} | Q_{API} | Q_{API} | Delta H |
| | J/g* | J/g* | J/g* | J/g** | kJ/mol |
| 10.4% loperamide base VP/AA | 263 | 202 | 61 | 586 | −279 |
| obtained from demin. water | | | | | |
| 32.92% loperamide base VP/AA | 154 | 151 | 3 | 8 | −4 |
| obtained from THF/methanol | | | | | |
| 10% loperamide/PVP K17 solid | 241 | 235 | 6 | 57 | −27 |
| solution | | | | | |
| Polymer VP/AA 80/20 | 226 | | | | |
| Kollidon ® 17 (PVP K17) | 261 | | | | |

*based on total initial weight
**based on proportion of active ingredient

Determination of the Loading Capacity of the Solid Solutions

1. A method of formulating a basic active ingredient sparingly soluble in water comprising adding a water-soluble polymer having a solubility in water of greater than 5% (m/m) in a pH range of 1 to 13 and which is obtained by a free-radically initiated polymerization of a monomer mixture of i) 70 to 90% by weight N-vinylpyrrolidone and ii) 10 to 30% by weight acrylic acid, wherein the sum total of i) and ii) corresponds to 100% by weight, to the basic active ingredient to provide a water-soluble salt, wherein the active ingredient in uncharged form or as hydrochloride has a solubility of less than 0.1% (m/m) in water, artificial intestinal juice, or gastric juice.

2. The method according to claim 1 for solubilizing the basic active ingredient.

3. The method according to claim 1, wherein the active ingredient has at least one and at most two groups capable of salt formation.

4. The method according to claim 1, wherein the water-soluble salt has a higher water solubility than the basic active ingredient and the corresponding hydrochloride of the basic active ingredient.

5. The method according to claim 1, wherein the water-soluble polymer has a solubility in water of greater than 10% (m/m) in the pH range of 1 to 13.

6. The method according to claim 1, wherein the water-soluble polymer in 5% by weight aqueous solution has a Fikentscher K-value of less than 30.

7. The method according to claim 1, wherein the water-soluble polymer in 5% by weight aqueous solution has a Fikentscher K-value of less than 20.

8. The method according to claim 1, comprising a water-soluble polymer which is obtained by free-radically initiated polymerization of a monomer mixture of i) 79 to 81% by weight N-vinylpyrrolidone and ii) 18 to 22% by weight acrylic acid, wherein the sum total of i) and ii) corresponds to 100% by weight.

9. The method according to claim 1, comprising a water-soluble polymer which is obtained by free-radically initiated polymerization of a monomer mixture of i) 80% by weight N-vinylpyrrolidone and ii) 20% by weight acrylic acid, wherein the sum total of i) and ii) corresponds to 100% by weight.

10. The method according to claim 1, wherein the formulation is in the form of a solid solution.

11. The method according to claim 1, wherein the water-soluble polymer acts as binder.

12. A dosage form comprising a water-soluble formulation of an active ingredient sparingly soluble in water and a polymer according to claim 1, wherein the formulation consists of a basic active ingredient, which in uncharged form or as the hydrochloride has a solubility of less than 0.1% (m/m) in water, artificial intestinal juice, or gastric juice, and a water-soluble polymer having a solubility in water of greater than 10% (m/m) in a pH range of 1 to 13 and which is obtained by a free-radically initiated polymerization of a monomer mixture of i) 78 to 82% by weight N-vinylpyrrolidone and ii) 18 to 22% by weight acrylic acid, wherein the sum total of i) and ii) corresponds to 100% by weight.

13. The dosage form according to claim 13, additionally comprising pharmaceutical auxiliaries.

14. The dosage form according to claim 13, prepared by compression.
